(19) United States
(12) Patent Application Publication
(10) Pub. No.: US 2013/0244960 A1
(43) Pub. Date: Sep. 19, 2013

(54) Treatment for Alzheimer's Disease

(71) Applicant: Kingsley Yianomah Quartey, Toronto (CA)

(72) Inventor: Kingsley Yianomah Quartey, Toronto (CA)

(21) Appl. No.: 13/421,744

(22) Filed: Mar. 15, 2012

(51) Int. Cl. A61K 31/7048; A61P 25/28; A61P 25/18; A61P 25/16

(52) U.S. Cl. 514/26

(57) ABSTRACT

Using a Pharmaceutically Suitable Preparation containing one more Cardio Glycosides such as THEVETIN B, THEVETIN A, PERUVOSIDE, THEVOFOLIN, OLEANDRIN, NERIIFOLIN, THEVETOXIN Derived and or Extracted from the Yellow Oleandrin Plant (Oleandrin Peruviana), White Oleandrin Plant (Nerium Oleander) and or other Plants in the Apocynaceae Genus, in the Dogbane Plant Family and or extracted from plants and or animals including plants and animals such as Foxglove or synthetic man-made, and or a combination of synthetic and natural thereof in Pharmaceutically Suitable Preparation in a Pharmaceutically Suitable Dose, example orally or intravenously, of which the Prescriber of the said Preparation, or Doctor, based on the overall health of the Patient will prescribe the best Dosage, The Best Mode of Dosage example orally or intravenously, which is best suited to have the Most Desirable Therapeutic Effect on the Patient

A Pharmaceutically acceptable Preparation containing one more Cardio Glycosides such as THEVETIN B, THEVETIN A, PERUVOSIDE, THEVOFOLIN, OLEANDRIN, NERIIFOLIN, THEVETOXIN Derived and or Extracted from the Yellow Oleander Plant (Thevetia Peruviana), White Oleander Plant (Nerium Oleander) and or other Plants in the Plantae Kingdom and Plantae Family of Apocynaceae, Genus in the Dogbane Plant Family and or extracted from plants and or animals including plants and animals such as Foxglove or Synthetic man-made Cardio Glycosides, and or a combination of synthetic and natural Cardio Glycosides such as THEVETIN B, THEVETIN A, PERUVOSIDE, THEVOFOLIN, OLEANDRIN, NERIIFOLIN, THEVETOXIN in Pharmaceutically Suitable Preparation for Treating:
(i) Alzheimer Disease
(ii) Parkinson Disease
(iii) Schizophrenia

With the said Pharmaceutically Suitable Preparation being Administered in a Pharmaceutically Acceptable Dosage, in a Pharmaceutically acceptable Mode and or manner, example, such as orally, or intravenously, and etcetera, with the mode and or method and of dosing and dosage depending on the Health and or Physical and Mental condition of the Patient being treated, the Health Care Practitioner and or the Doctor Prescribing the Said Pharmaceutically Suitable Preparation made with and or Containing Cardio Glycosides for Treating:
(i) Alzheimer Disease
(ii) Parkinson Disease
(iii) Schizophrenia

Will choose and or Prescribe the method or dosage and dose form, such as for example, orally or intravenously to have the most desired therapeutic effect on the Patient being treated

### BACKGROUND OF INVENTION

### Prior Art

Prior to my Invention, there was no effective Treatment for:
(i) Alzheimer Disease
(ii) Parkinson Disease
(iii) Schizophrenia

That was effective in Treating (i) Alzheimer Disease, (ii) Parkinson Disease and (iii) Schizophrenia. All this three diseases and or disorders create enormous burdens of Families, Societies, and the Sufferers of the Diseases themselves.

And those that have (i) Alzheimer Disease, (ii) Parkinson Disease (iii) Schizophrenia are in what is essentially a Mental and or Neurological Debilitated State and Prior to my Invention there is no effective Treatment and or Cure for this debilitating disorders.

(i) Alzheimer Disease:

In Healthy Brains, Proteins in the Brains call Tau helps transport nutrients and other important substances from one nerve cell to another in the brains, However, With Alzheimer Disease abnormal Taus proteins, Prions such as Beta-Amyloid Plaques and neurofibrillary tangles and etcetera over-accumulates in the Brains of an Individual, the Individual develops a Neurodegenerative Disease that is known as Alzheimer Disease.

The Taus and or Prions such as Beta-Amyloids forms insoluble messy plaques and tangles in the brains of those that suffers from Alzheimer Disease, impairing their Neurocognitive

Abilities, impairing their Memories, Causing Severe Memory Loss, impairing their Motor skills and in most cases making simple everyday functions that is normal part of life impossible.

While there are medications that are helpful in reducing some of the symptoms Alzheimer Disease in some Individuals Suffering from Alzheimer Disease, this medications do not remove or reduces the plaques in the brains that causes Alzheimer Disease

Prior to my Invention there was no Effective Cure or Treatment for Alzheimer Disease.

There was no effective Drug or Treatment that removes the insoluble plagues, proteins and tangles that forms in the brains of those that have the Alzheimer Disease, and those plagues in the brains that causes Alzheimer Disease cannot be surgically removed without killing or permanently maiming the Individual suffering from the Alzheimer Disease

(ii) Parkinson Disease

Parkinson Disease is a Neurodegenerative disorder of the central nervous system. It causes characteristic motor problems, such as gait, involuntary shakiness, difficulty in walking and talking, and host of other neurological and psychiatric problems. While it is idiopathic, meaning there is no known cause, it is believed to be caused by genetic and environmental conditions, example, consistent blows to the head is one of the suspected causes, and death of dopamine cells in the brains and abnormal aggregation of spherical mass proteins in the brains called Lewy Bodies.

This Lewy Bodies displaces other cell components in the brains, has also being cited as potential cause of Parkinson Disease, or at least the Lewy Bodies helps creates the condition in the brain that helps cause the disorder that is known as Parkinson Disease.

There are drugs that help to alleviate some of the symptoms in some of the Individuals suffering from Parkinson Disease, however, there is no cure.

Prior to my Novel Invention which I can demonstrate in any forum if required, there was no known cure for Parkinson Disease

(iii) Schizophrenia:

Schizophrenia is a Psychiatric Disorder that is caused by environmental factors and genetics.

Prior to my Novel Invention which I can demonstrate in any appropriate forum if required or requested to do so, there was no effective Treatment for Schizophrenia other than Antipsychotic medications such as “Clozapine”, which itself has serious side effects such as reducing the white blood cells of the person taking it, thereby increasing the person's risk for infection. Other Antipsychotic drugs cause Muscle Spasms, in some instances and Menstrual Problems in Women.

The anti-psychotic drugs are often Symptoms Targeted, example if someone with Schizophrenia is having hallucinations, they are given a antipsychotic drugs that deals with hallucination, however, there is no drug or treatment that stops or effectively treat Schizophrenia so that is cured or it is minimized to the extent that the person Suffering from Schizophrenia can lead a normal life without medication.

Prior to my novel Invention, which I can demonstrate as an effective Treatment for Schizophrenia in any forum if required to do so, prior to my Invention there was no effective Treatment for Schizophrenia.

### BRIEF SUMMARY OF THE INVENTION

(i) Treatment for Alzheimer Disease

(ii) Treatment for Parkinson Disease

(iii) Treatment for Schizophrenia

a Pharmaceutically Suitable Preparation containing one more Cardio Glycosides such as THEVETIN B, THEVETIN A, PERUVOSIDE, THEVOFOLIN, OLEANDRIN, NERIIFOLIN, THEVETOXIN Derived and or Extracted from the Yellow Oleandrin Plant (Thevetia Peruviana), White Oleandrin Plant (Nerium Oleander) and or other Plants in the Plantae Kingdom and Plantae Family of Apocynaceae, Genus in the Dogbane Plant Family or other Plants in the Apocynaceae Genus, in the Dogbane Plant Family and or extracted from plants and or animals including plants and animals such as Foxglove or synthetic man-made, and or a combination of synthetic and natural Cardio Glycosides such as THEVETIN B, THEVETIN A, PERUVOSIDE, THEVOFOLIN, OLEANDRIN, NERIIFOLIN, THEVETOXIN thereof in Pharmaceutically Suitable Preparation for Treating:
(i) Alzheimer Disease
(ii) Parkinson Disease
(iii) Schizophrenia

### Alzheimer Disease:

My Novel Invention helps to reduces and or removes the Plaques and Tangles in the Brains that causes the disorder known as Alzheimer's Disease My Novel Invention also helps promote a healthier environment in the Brain, this helps Treat and Ameliorate Neurological Disorders in the brain

### Parkinson Disease:

My Novel Invention reduces and or removes the Lewy Bodies that helps causes the disorder known as PARKINSON DISEASE

My Novel Invention also helps promote a healthier environment in the Brain, this helps Treat and Ameliorate Neurological Disorders in the brain

and helps promote a healthier environment in the Brain, this helps Treat and Ameliorate Neurological Disorders in the brain.

### Schizophrenia:

My Novel Invention works by Creating a Healthier Environment In the Brain, thereby Treating and Effectively Curing Neurodegenerative and Psychiatric Disorders, including Schizophrenia

### DETAILED DESCRIPTION OF THE INVENTION

(i) Treatment for Alzheimer Disease

(ii) Treatment for Parkinson Disease

(ii) Treatment for Schizophrenia

My Novel Invention helps:

Reduces and or removes the Tau Plaques, and or Beta-Amyloids and Tangles in the Brains of Individuals suffering from Alzheimer's Disease

Reduces and or removes the Lewy Bodies that helps causes the disorder known as PARKINSON DISEASE

By getting rid of the accumulants and abnormal proteins in the brain, My Novel Invention creates a Healthier Environment In the Brain, thereby Effectively Treating and Effectively Curing Neurodegenerative Diseases and also Psychiatric Disorders, especially

(i) Alzheimer's Disease, (ii) Parkinson Disease (iii) Schizophrenia

By using a Pharmaceutically Suitable Preparation containing one more Cardio Glycosides such as THEVETIN B, THEVETIN A, PERUVOSIDE, THEVOFOLIN, OLEANDRIN, NERIIFOLIN, THEVETOXIN Derived and or Extracted from the Yellow Oleandrin Plant (Thevetia Peruviana), White Oleander Plant (Nerium Oleander) and or other Plants in the Apocynaceae Genus, in the Dogbane Plant Family and or extracted from plants and or animals including plants and animals such as Foxglove or synthetic man-made, and or a combination of synthetic and natural Cardio Glycosides such as THEVETIN B, THEVETIN A, PERUVOSIDE, THEVOFOLIN, OLEANDRIN, NERIIFOLIN, THEVETOXIN thereof in Pharmaceutically Suitable Preparation for Treating:
(i) Alzheimer Disease
(ii) Parkinson Disease
(iii) Schizophrenia

The Healthcare Practitioner and or Doctor, based on the overall health of the Patient will prescribe the best Dosage, Mode of Dosage example orally or intravenously, best suited to have the Most Desirable Therapeutic Effect on the Patient

What is claimed is:

i) Treatment for Alzheimer's Disease
Using a Pharmaceutically Suitable Preparation containing one more Cardio Glycosides such as THEVETIN B, THEVETIN A, PERUVOSIDE, THEVOFOLIN, OLEANDRIN, NERIIFOLIN, THEVETOXIN Derived and or Extracted from the Yellow Oleandrin Plant (Thevetia Peruviana), White Oleandrin Plant (Nerium Oleander) and or other Plants in the Apocynaceae Genus, in the Dogbane Plant Family and or extracted from plants and or animals including plants and animals such as Foxglove or synthetic man-made, and or a combination of synthetic and natural Cardio Glycosides such as THEVETIN B, THEVETIN A, PERUVOSIDE, THEVOFOLIN, OLEANDRIN, NERIIFOLIN, THEVETOXIN thereof in a Pharmaceutically Suitable Preparation in a Pharmaceutically Suitable Dose, and Pharmaceutically Dosage form, example orally or intravenously, of which the Prescriber of the said Preparation, such as the Health Care Practitioner and or Doctor, based on the overall health of the Patient will prescribe the best Dosage, The Best Mode of Dosage example orally or intravenously, which is best suited to have the Most Desirable Therapeutic Effect on the Patient as my Invention.

ii) Treatment for Parkinson's Disease
Using a Pharmaceutically Suitable Preparation containing one more Cardio Glycosides such as THEVETIN B, THEVETIN A, PERUVOSIDE, THEVOFOLIN, OLEANDRIN, NERIIFOLIN, THEVETOXIN Derived and or Extracted from the Yellow Oleandrin Plant (Thevetia Peruviana), White Oleandrin Plant (Nerium Oleander) and or other Plants in the Apocynaceae Genus, in the Dogbane Plant Family and or extracted from plants and or animals including plants and animals such as Foxglove or synthetic man-made, and or a combination of synthetic and natural Cardio Glycosides such as THEVETIN B, THEVETIN A, PERUVOSIDE, THEVOFOLIN, OLEANDRIN, NERIIFOLIN, THEVETOXIN thereof in a Pharmaceutically Suitable Preparation in a Pharmaceutically Suitable Dose, and Pharmaceutically Dosage form, example orally or intravenously, of which the Prescriber of the said Preparation, such as the Health Care Practitioner and or Doctor, based on the overall health of the Patient will prescribe the best Dosage, The Best Mode of Dosage example orally or intravenously, which is best suited to have the Most Desirable Therapeutic Effect on the Patient as my Invention.

iii) Treatment for Schizophrenia Disorder
Using a Pharmaceutically Suitable Preparation containing one more Cardio Glycosides such as THEVETIN B, THEVETIN A, PERUVOSIDE, THEVOFOLIN, OLEANDRIN, NERIIFOLIN, THEVETOXIN Derived and or Extracted from the Yellow Oleandrin Plant (Thevetia Peruviana), White Oleandrin Plant (Nerium Oleander) and or other Plants in the Apocynaceae Genus, in the Dogbane Plant Family and or extracted from plants and or animals including plants and animals such as Foxglove or synthetic man-made, and or a combination of synthetic and natural Cardio Glycosides such as THEVETIN B, THEVETIN A, PERUVOSIDE, THEVOFOLIN, OLEANDRIN, NERIIFOLIN, THEVETOXIN thereof in a Pharmaceutically Suitable Preparation in a Pharmaceutically Suitable Dose, and Pharmaceutically Dosage form, example orally or intravenously, of which the Prescriber of the said Preparation, such as the Health Care Practitioner and or Doctor, based on the overall health of the Patient will prescribe the best Dosage, The Best Mode of Dosage example orally or intravenously, which is best suited to have the Most Desirable Therapeutic Effect on the Patient as my Invention.
